(19) United States
(12) Patent Application Publication
(10) Pub. No.: US 2004/0087644 A1
(43) Pub. Date: May 6, 2004

(54) Pharmaceutical composition in capsules that comprises a non-steroidal antiinflamatory and an optiate analgesic for handling pain

(75) Inventors: Maria Elena Garcia Armenta, Zapopan (MX); Victor Guillermo Alvarez Ochoa, Zapopan (MX); Josefina Santos Murillo, Zapopan (MX)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Leopoldo Espinosa Abdala, Guadalajara (MX)

(21) Appl. No.: 10/602,190

(22) Filed: Jun. 24, 2003

(30) Foreign Application Priority Data
Nov. 4, 2002 (MX) PA/A/2002/010828

(51) Int. Cl. A61K 31/407; A61K 31/137

(52) U.S. Cl. 514/412; 514/650

(57) ABSTRACT

The present invention is referred as a formula or combination of two compounds, one of them being a non-steroidal anti-inflammatory such as ketorolac, and the other an opiate analgesic known as tramadol, which are formulated in capsules.

The combination of these compounds produces a greater analgesic effect, with less dosage and less side effects for the treatment of pain.

### FIELD OF THE INVENTION

The present invention is referred as the association or combination of a non-steroidal anti-inflammatory agent such as ketorolac and an opiate analgesic known as tramadol; which are formulated into capsules and that are given to patients who have pain.

The combination of these substances, gives as a result a greater analgesic effect, with an analgesic synergy, as opposed to when these substances are independently administered. The dosage is also less, avoiding side effects when other methods of administration are used or when they are independently used.

### BACKGROUND OF THE INVENTION

Ketorolac is a non-steroidal anti-inflammatory agent with analgesic properties. In the case of pain, it can be administered orally or by injection (intramuscularly or intravenously). It has been shown that Ketorolaco shows an analgesic efficiency comparable with opiates, according to clinical studies reported by Yee et al in 1986; O'Hara et al in 1987, and Forbes et al in 1990.

It has been shown that Ketorolac acts over the ciclo-oxygenase enzyme, which acts on the pain and inflammatory process, it has a plasmatic half-life of 4 to 6 hours. About 90% of the dose is excreted through the urine without changes; the rest is excreted through the feces.

Tramadol is an opiate analgesic, it has noradrenergic and serotoinergic properties that contribute in its analgesic activities. It is used to moderate severe pain. It can be administered orally, intramuscularly or intravenously. Tramadol half-life is 6 hours and is mainly excreted through the urine.

Our interest was to create a combination of a non-steroidal anti-inflammatory with analgesic activity as ketorolac and of an opiate analgesic such as tramadol, with less dosage, to be administered orally, avoiding the typical side effects when administered independently and with greater dosage.

There was an unexpected side effect between the two active agents administered together, differently than those obtained when administered independently, furthermore we also used less dosage without side effects.

### DETAILED DESCRIPTION OF THE INVENTION

In the last couple of years has increased the associative research between analgesic medicines, with the objective of create an analgesic synergy, using less dosage and reducing the side effects of those obtained when administered independently, using greater dosages.

A clinical study was performed, using the combination of ketorolac/tramadol capsules in 100 patients suffering postoperative pain because the second molar extraction.

The patients fulfilled a questionnaire, with the objective of determining the efficacy of the ketorolac/tramadol combination. A visual analog scale (VAS) was applied to them to measure the pain intensity, before and after the administration of the combination, the initiation time was measured until 15 and 30 minutes after administration and side effects were observed. The results are the following:

TABLE 1
MEASUREMENT OF PAIN BEFORE ADMINISTRATION

| Symptoms | No. OF PATIENTS |
|---|---|
| Pain | |
| Absent | 0 |
| Low | 25 |
| Moderate | 50 |
| Severe | 25 |

TABLE 2
MEASUREMENT OF PAIN 15 MINUTES AFTER
ADMINISTRATION

| SYMPTOMS | No. of Patients |
|---|---|
| Pain | |
| Absent | 75 |
| Low | 20 |
| Moderate | 5 |
| Severe | 0 |

TABLE 3
MEASUREMENT OF PAIN 30 MINUTES AFTER
ADMINISTRATION

| Symptoms | No. of Patients |
|---|---|
| Pain | |
| Absent | 80 |
| Low | 18 |
| Moderate | 2 |
| Severe | 0 |

Side effects were not reported during and after the administration.

MEASUREMENT OF PAIN 45 MINUTES AFTER
ADMINISTRATION

| Symptoms | No. of Patients |
|---|---|
| Pain | |
| Low | 81 |
| Moderate | 19 |
| Severe | 0 |

Side effects were not reported during and after the administration.

| Composition: | |
|---|---|
| Ketorolaco Tromethamine | from 0.0010 g to 0.10000 g |
| Tramadol hydrochloride | from 0.0001 g to 0.20000 g |
| Colloidal silicon dioxide | from 0.0001 g to 0.20000 g |
| Sodium glicolate starch | from 0.010 g to 0.20000 g |
| Lactose | from 0.0100 g to 0.50000 g |
| Microcrystalline cellulose | from 0.0100 g to 0.5000 g |
| Magnesium estearate | from 0.0001 g to 0.02000 g |
| Excipients | from 0.0001 g to 1.0000 g |

Elaboration Process

1. Mix the ketorolac tromethanine, the colloidal silicate dioxide, the tramadol hydrochloride, sodium glicolate starch, the lactose, the microcrystalline cellulose, the magnesium stearate and other recipients if necessary

2. Analyze the powdered mix

3. Proceed to encapsuling and conditioning

It is important to notice that the combination of the active principles stated above offer great advantages, differently than those obtained when administered independently. In the combination of said principles the dose is less and the efficacy is excellent. This combination results in a reduction of side effects of those obtained when the active principles are administered independently and in greater doses.

### NOVELTY OF THE INVENTION

Having described the present invention, it is considered a novelty and as a result it is claimed as property the following:

1. A pharmaceutical composition, comprised of a non-steroidal anti-inflammatory and an opiate analgesic in combination with colloidal silicate dioxide, sodium glicolate, lactose, microcrystalline cellulose, magnesium estearate and other recipients if necessary.

2. A pharmaceutical composition according to claim 1, wherein the non-steroidal anti-inflammatory is ketorolac tromethamine and the opiate analgesic is tramadol hydrochloride.

3. A pharmaceutical composition according to claim 1, wherein the ketorolac tromethamine is present in the composition in a range of 0.0010 g to 0.1000 g and the tramadol hydrochloride is present in a range of 0.0010 g to 0.2000 g.

4. A pharmaceutical composition according to claim 1, wherein the ketorolaco tromethamine is present in the composition in a range of 0.0010 g to 0.1000 g and the tramadol hydrochloride is present at a range of 0.0010 g to 0.2000 g, the colloidal silicate dioxide is present in a range of 0.00010 g to 0.02000 g, the sodium glicolato starch is present in a range of 0.0010 g to 0.2000 g, the lactose is present in a range of 0.0100 g to 0.5000 g, the microcrystalline cellulose is present in a range of 0.0100 g to 0.5000 g, the magnesium stearate is present at a proportion of 0.0001 g to 0.02000 g, other recipients can be present at a proportion of 0.0001 g to 1.0000 g.

5. A pharmaceutical composition according to claim 1, wherein it is in a capsule form.

6. A procedure for the elaboration of a composition according to claim 1, wherein it is comprised of the following steps:
a) Mix the ketorolac tromethanine, the colloidal silicate dioxide, the tramadol hydrochloride, sodium glicolate starch, the lactose, the microcrystalline cellulose, the magnesium stearate and other recipients if necessary ; b) analyze the powdered mix; c) proceed to encapsuling and conditioning the mix.

7. The use of the composition according to claims 1 to 6, for the pain treatment.
